(19) United States
(12) Patent Application Publication
(10) Pub. No.: US 2025/0352320 A1
(43) Pub. Date: Nov. 20, 2025

(54) GYNECOLOGICAL PROTHETIC WITH ANCHOR MEMBERS AND EXPANDABLE CONNECTING LINKS, AND METHOD FOR CUSTOM-DESIGNNG THE SAME

(71) Applicant: FEMTHERAPEUTICS INC., Montreal (CA)

(72) Inventors: Negin ASHOURI, Montreal (CA); Inara LALANI, Montreal (CA); Juliette JAHANGARD, Montreal (CA); Mihnea GANGAL, Montreal (CA); Majid ROSHANFAR, Montreal (CA); Erfan FATEHI, Montreal (CA); Lydia AGUIRRE PERALES, Montreal (CA); Prabhakara JOIS, Montreal (CA)

(21) Appl. No.: 18/862,715

(22) PCT Filed: May 6, 2022
(86) PCT No.: PCT/CA2022/050721

§ 371 Date: Nov. 4, 2024

(51) Int. Cl. A61F 2/04; B33Y 80/00

(52) U.S. Cl. CPC A61F 2/04; B33Y 80/00; A61F 2240/002

(57) ABSTRACT

Disclosed is a gynecological prosthetic including a plurality of anchor members configured to rest on anchor points within a vagina, and a plurality of connecting links that couple the anchor members together. The connecting links are configured to be compressible to enable a compact state when the gynecological prosthetic is being installed in the vagina. Also, the connecting links are configured to be expandable from the compact state after the gynecological prosthetic is installed in the vagina so as to push the anchor members outward against inside surfaces of the vagina to resist displacement of the gynecological prosthetic. This gynecological prosthetic can have a better fit to an anatomical shape of an upper vagina compared to current gynecological prosthetics, in part because its design and flexibility enable it to expand in certain directions. Also disclosed is a method and apparatus for custom-designing the gynecological prosthetic.

### FIELD OF THE DISCLOSURE

This disclosure relates to gynecological prosthetics, and more particularly to gynecological prosthetics that are custom-fitted to treat pelvic floor disorders while mitigating complications.

### BACKGROUND

One in every four women, and one in every two women over 80, may suffer from Pelvic Floor Disorders (PFD's), such as fecal and urinary incontinence and Pelvic Organ Prolapse (POP). POP is a condition that involves an abnormal descent of pelvic organs beyond vaginal walls. Symptoms include vagina discomfort, leakage of urine, and bowel problems—all of which can affect a woman's quality of life drastically. Vaginal childbirth and menopause are the two leading causes of POP, making it virtually certain that every woman has at least one hash mark on her risk factor profile for this complication.

Research indicates that in the United States alone there are approximately 3.3 million women with POP. These disorders are severely underserved and produce a significant gender disparity in medical innovation. The dynamic behind POP is likely more diverse than any other health condition women will experience due to differences in genetics, general health, vaginal childbirth, occupation and social lifestyle.

There are non-surgical and surgical treatment options for POP. Non-surgical treatment options include Kegel exercises, Kegel assist devices, gynecological prosthetics, core/floor strengthening exercises, biofeedback, electrical stimulation, hormone replacement therapy, and support garments. Regarding surgery, approximately 20% of patients will undergo pelvic floor surgical procedures which carry life-long risks including incontinence, inability to child-bare, pain during sex, and approximately 30% chance of developing another prolapse or other complications. There have also been several reported risks of complications with transvaginal mesh implants which have since been based by the FDA in April 2019. Approximately 300,000 surgeries are performed annually in the United States. These surgeries are very costly and may not be accessible to patients that have high comorbidities.

Gynecological prosthetics, such as pessaries or other therapeutic prosthetic devices, are intravaginal devices that are used to provide support and reduce symptoms of prolapse and urinary/fecal incontinence. However, research shows that around 56% of patients will develop complications with long-term use such as irritation, lacerations, open sores, displacement and discharge, and chronic pain. In fact, about one in three women will fail prosthetic fittings, and approximately 50% of women will discontinue use after a year and ultimately resort to high-risk and expensive surgical procedures. Current methods of choosing a prosthetic type and size involve manual measurements and the Pelvic Organ Prolapse Quantification system (POP-Q). POP-Q refers to an objective, site-specific system for describing, quantifying, and staging pelvic support in women.

Gynecological prosthetics have high failure rates due to the design of the gynecological prosthetics, the management of the gynecological prosthetics and their fit. There are over 100+ shapes and sizes of gynecological prosthetics which are designed with simple geometric and symmetrical shapes, such as cubes, and donuts. Current producers of gynecological prosthetics fail to consider that multiple types of POP display a variety of symptoms and that every woman experiences unique childbirth due to their genetics and general health. Studies show that approximately 90% of patients can be treated with a non-pharmaceutical and/or non-surgical solution if there was an effective method on the market.

There is a clear unmet need in the market to improve current gynecological prosthetics and prevent surgical intervention. It is an object of the disclosure to improve upon the conventional approaches to address or mitigate some or all of the shortcomings noted above.

### SUMMARY OF THE DISCLOSURE

Disclosed is a gynecological prosthetic including a plurality of anchor members configured to rest on anchor points within a vagina, and a plurality of connecting links that couple the anchor members together. The connecting links are configured to be compressible to enable a compact state when the gynecological prosthetic is being installed in the vagina. Also, the connecting links are configured to be expandable from the compact state after the gynecological prosthetic is installed in the vagina so as to push the anchor members outward against the inside surfaces of the vagina to resist displacement of the gynecological prosthetic.

In some implementations, the anchor members and the connecting links form a base plate, and the gynecological prosthetic also has an upper plate coupled to the base plate via upper links that are positioned such that, when a force is applied to the upper plate, the upper links redirect at least some of that force to push the anchor members of the base plate outward against the inside surfaces of the vagina to further resist displacement of the gynecological prosthetic.

This gynecological prosthetic can have a better fit to an anatomical shape of an upper vagina compared to current gynecological prosthetics, in part because its design and flexibility enable it to expand in certain directions and rest on the anchor points in the vaginal canal. This can redirect pressure from the anterior and posterior walls of a vaginal canal to side walls. In some implementations, the gynecological prosthetic is designed to rest on some parts of the iliococcygeus and pubococcygeus muscles.

Also disclosed is a gynecological prosthetic configured to rest on anchor points within a vaginal canal. In some implementations, the gynecological prosthetic has geometry and/or materials designed based on a cervical angle.

Also disclosed is a method of custom-designing a gynecological prosthetic as summarized above. The method involves acquiring, by a processor, a patient data pertaining to a subject, selecting the gynecological prosthetic out of a plurality of pre-defined gynecological prosthetics, wherein each pre-defined gynecological prosthetic is configured to expand differently and in different directions based on received pressure from surrounding tissues and organs, and calculating, by the processor, geometry and/or material of the gynecological prosthetic that has been selected based on the patient data that has been acquired. In this way, a gynecological prosthetic can be selected and customized based on characteristics of the subject, such as the size of the upper vagina, and the gynecological prosthetic can be tailored specifically to the subject's anatomy and body habitus.

Also disclosed is a non-transitory computer readable medium having recorded thereon statements and instructions that, when executed by a processor of a computing device, configure the processor to implement a method as summarized above.

Also disclosed is a computing device having a processor, and a non-transitory computer readable medium having recorded thereon statements and instructions that, when executed by the processor, configure the processor to implement a method as summarized above.

Also disclosed is a manufacturing method. The manufacturing method involves 3D printing a gynecological prosthetic using PolyJet technology to combine at least two polymers in the gynecological prosthetic, and then coating the gynecological prosthetic with a medical grade silicon.

Also disclosed is a kit having a gynecological prosthetic as summarized above, and an applicator configured to hold the gynecological prosthetic in the compact state while being inserted into a vagina and to release the gynecological prosthetic into the vagina for installation.

Also disclosed is a method of installing a gynecological prosthetic as summarized above. The method involves loading the gynecological prosthetic into an applicator configured to hold the gynecological prosthetic in the compact state, inserting the gynecological prosthetic into a vagina using the applicator, and releasing the gynecological prosthetic from the applicator thereby installing the gynecological prosthetic in the vagina.

Other aspects and features of the present disclosure will become apparent, to those ordinarily skilled in the art, upon review of the following description of the various embodiments of the disclosure.

### BRIEF DESCRIPTION OF THE DRAWINGS

Embodiments will now be described with reference to the attached drawings in which:

FIGS. 1A and 1B are schematics of a vagina in which a gynecological prosthetic has been installed, in accordance with an embodiment of the disclosure;

FIGS. 2A to 2E are schematics of the gynecological prosthetic of FIGS. 1A and 1B;

FIGS. 3A to 3C are schematics of another gynecological prosthetic, in accordance with an embodiment of the disclosure;

FIGS. 4A to 4D are schematics of another gynecological prosthetic, in accordance with an embodiment of the disclosure;

FIG. 5 is a block diagram of a gynecological prosthetic customization system having a computing device 510;

FIG. 6 is a flowchart of a method of custom-designing a gynecological prosthetic;

FIGS. 7A and 7B are flowcharts of another method of custom-designing a gynecological prosthetic;

FIG. 8 is a schematic of a vaginal canal simulated model and shows where a gynecological prosthetic may rest;

FIGS. 9A and 9B are schematics of a pelvic bony structure including a top view and a bottom view:

FIG. 10 is a schematic with variables measured using the POP-Q measurement system;

FIG. 11 is a graph of a cervical angle vs. position within the vagina;

FIG. 12 is a flowchart of an exemplary process for providing a user with a custom therapeutic device, in accordance with an embodiment of the disclosure;

FIG. 13 is a schematic of an applicator for installing a gynecological prosthetic; and

FIG. 14 is a flowchart of a method of installing a gynecological prosthetic.

### DETAILED DESCRIPTION OF EMBODIMENTS

It should be understood at the outset that although illustrative implementations of one or more embodiments of the present disclosure are provided below, the disclosed systems and/or methods may be implemented using any number of techniques. The disclosure should in no way be limited to the illustrative implementations, drawings, and techniques illustrated below, including the exemplary designs and implementations illustrated and described herein, but may be modified within the scope of the appended claims along with their full scope of equivalents.

### Example Gynecological Prosthetics

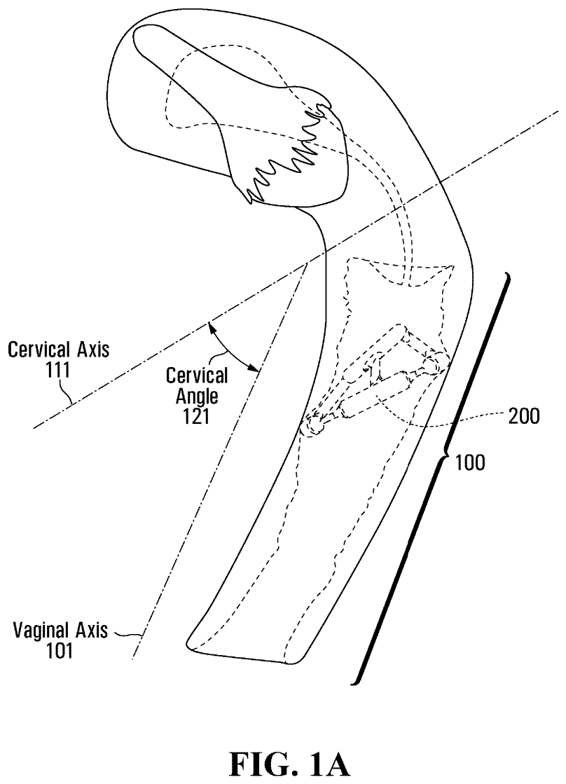

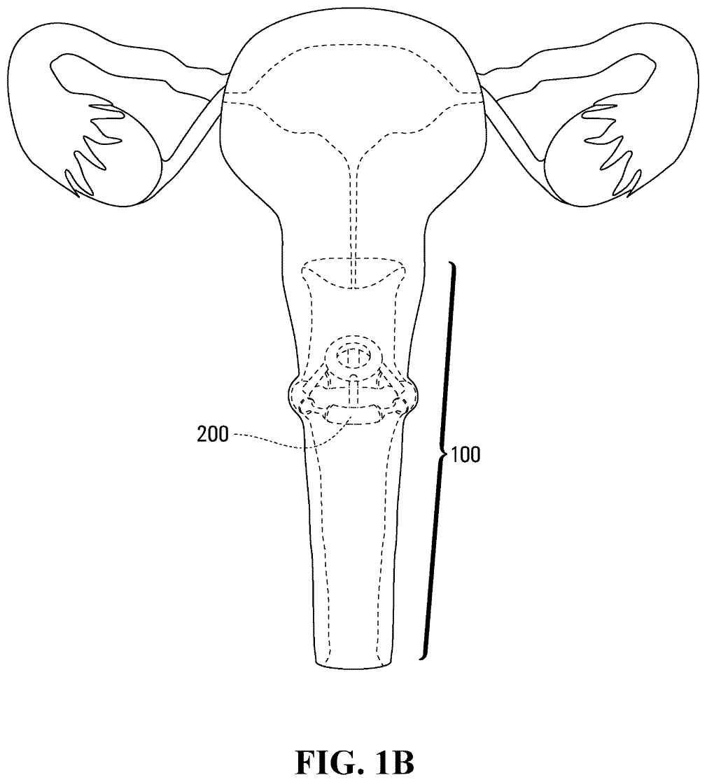

Referring first to FIGS. 1A and 1B, shown are schematics of a vagina 100 in which a gynecological prosthetic 200 has been installed, in accordance with an embodiment of the disclosure. The gynecological prosthetic 200 can be installed in the vagina 100 to treat Pelvic Organ Prolapse (POP), by providing support for pelvic organs (not shown) and thereby avoid or mitigate abnormal descent of the pelvic organs beyond vaginal walls. Additionally, or alternatively, the gynecological prosthetic 200 can be installed in the vagina 100 to treat urinary and/or fecal incontinence. In some implementations, the gynecological prosthetic 200 is positioned in an upper portion of the vagina 100 and rests on iliococcygeus and pubococcygeus muscles (not shown), which serve as anchor points within the vagina 100. However, other positions within the vagina 100 are possible, such that the gynecological prosthetic 200 can sit anywhere in the vaginal canal, and this may depend on the patient's anatomical characteristics and which pelvic organs are being targeted. Also shown is cervical angle 121 between a vaginal axis 101 and a cervical axis 111.

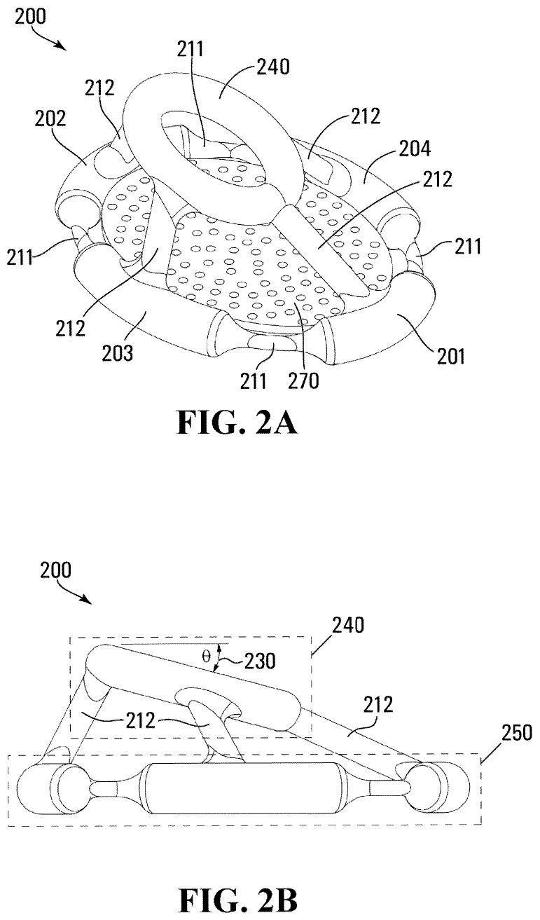

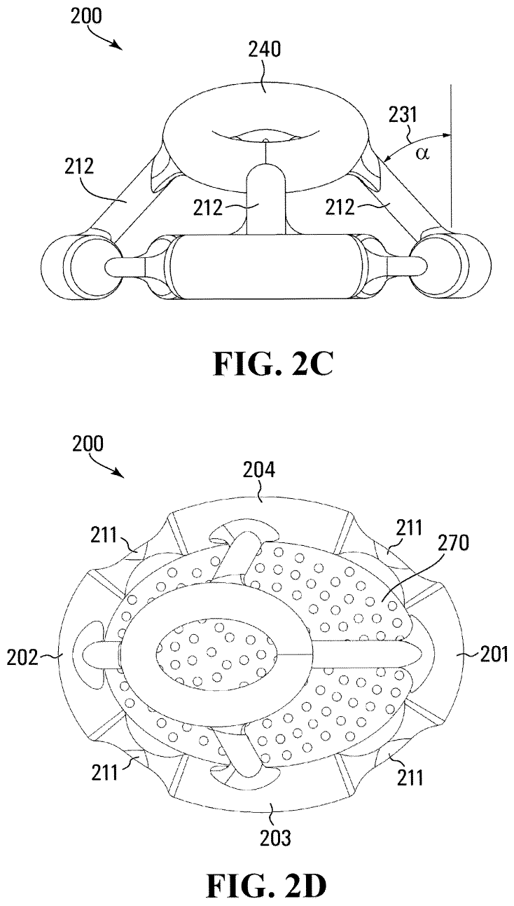

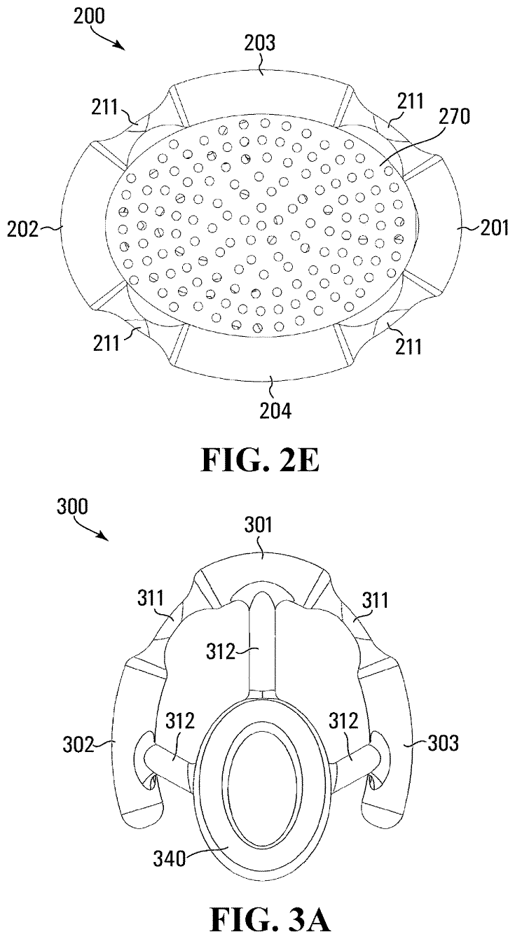

Referring now to FIGS. 2A to 2E, shown are schematics of the gynecological prosthetic 200 of FIGS. 1A and 1B. The gynecological prosthetic 200 has a plurality of anchor members 201-204 configured to rest on the anchor points within the vagina 100, and a plurality of connecting links 211 that couple the anchor members 201-204 together. The connecting links 211 are configured to be compressible to enable a compact state when the gynecological prosthetic 200 is being installed in the vagina 100. Also, the connecting links 211 are configured to be expandable from the compact state after the gynecological prosthetic 200 is installed in the vagina 100 so as to push the anchor members 201-204 outward against inside surfaces of the vagina 100 to resist displacement of the gynecological prosthetic 200. The connecting links 211 are designed to provide flexibility and connect the anchor members 201-204 and prevent them from bending or deformation. The anchor members 201-204 are configured to rest on the anchor points within the vagina 100, and are designed to apply pressure on surrounding muscles and tissues in order to help support the pelvic organs and to hold the whole gynecological prosthetic 200 in place.

In some implementations, the anchor members 201-204 and the connecting links 211 form a base plate 250, and the gynecological prosthetic 200 also has an upper plate 240 coupled to the base plate 250 via a plurality of upper links 212. In some implementations, the upper links 212 can be compressed (or rotated, etc.) to make insertion of the gynecological prosthetic 200 into the vagina 100 possible. In some implementations, the upper plate 240 is to be positioned under the uterus such as a cervix area to hold the uterus and provide support to cervix area. In some implementations, the base plate 250 is designed to provide support to the whole pelvic area and prevent the pelvic organs from descending. The upper links 212 are positioned such that, when a force is applied to the upper plate 240, the upper links 212 redirect at least some of that force to push the anchor members 201-204 of the base plate 250 outward against the inside surfaces of the vagina 100 to further resist displacement of the gynecological prosthetic 200.

Therefore, any downward force that may be applied to the upper plate 240 of the gynecological prosthetic 200 can contribute to the anchor members 201-204 being pushed outward to resist displacement of the gynecological prosthetic 200. The upper plate 240 and its upper links 212 can redistribute pressure toward a white line (tendinous arch), which is a medical term to name the thickening of the parietal fascia of levator ani muscles along the straight line from the pubic arch to the ischial spine bilaterally. The manner in which the anchor members 201-204 are pushed outward will depend on many factors such as the anchor members 201-204, the upper plate 240, the connecting links 211, the upper links 212, overall geometry of the gynecological prosthetic 200, materials used, and the downward and surrounding pressures such as abdominal pressure or intravaginal pressure in each patient. The upper plate 240 can be positioned at any suitable distance from the base plate 250 and its position and size can vary for each patient.

In some implementations, the upper plate 240 is smaller than the base plate and the upper links 212 connect a periphery of the upper plate to a periphery of the base plate 250. Thus, each upper link 212 is tilted towards the upper plate 240 at an angle 231, which enables any downward and surrounding force that may be applied to the upper plate 240 of the gynecological prosthetic 200 to contribute to the anchor members 201-204 being pushed outward. Note that force might not only come from above the upper plate 240, but also it may come from the anterior (e.g. bladder) or posterior (e.g. rectum) walls as well as from apical (e.g. uterus), so this might also have an effect not only on the upper plate 240 but also on one of more of the upper links 212. Nonetheless, forces can be redistributed to the lateral walls. The angle 231 can be different for each upper link 212 and can for example include any suitable value in a range between α=0° to 90°.

In some implementations, the anchor members 201-204 include an anterior member 201 configured to rest on an anterior wall of the vagina 100, a posterior member 202 configured to rest on a posterior wall of the vagina 100, and side members 203 and 204 configured to rest on lateral walls of the vagina 100. The anterior and posterior members 201 and 202 can provide outward force against the anterior and posterior walls of the vagina 100. Meanwhile, the side members 203 and 204 can provide outward force against the side walls of the vagina 100. The amount of force for each anchor member 201-204 can be designed by geometry, material and force of the gynecological prosthetic 200, for example via the angle 231 of each link 212. The anchor members 201-204 can thus distribute pressure among the anterior, posterior and lateral walls of the vagina 100. Although four anchor members 201-204 are shown, it is noted that other implementations are possible in which more or fewer anchor members are present. Such anchor members can distribute pressure among the lateral walls of the vagina via side members, and among the anterior and/or posterior walls of the vagina depending on whether anterior and posterior members are present.

In some implementations, the upper plate 240 is tilted by an angle 230 relative to the base plate 250, such that the upper plate 240 and the base plate 250 are not parallel to one another. This tilt can be designed based on geometry and/or characteristics of the vagina 100, such that the upper plate 240 is suitably positioned to transfer any downward or surrounding force to the anchor members 201-204. In some implementations, the angle 230 corresponds to the cervical angle 121 between the cervical axis 111 and the vaginal axis 101. The angle 230 can for example be between θ=0° to 80°, with an anterior portion of the upper plate 240 being lowered towards the base plate 250. However, for abnormal situations in which the uterus is positioned in the opposite direction to what is shown in FIG. 1A, the upper plate 240 can be designed with a tilt in the opposite direction to what is shown in FIG. 2B, such that the angle 230 is between θ=−80° to 0°, with the anterior portion of the upper plate 240 being raised away from the base plate 250. In other implementations, the upper plate 240 is parallel to the base plate 250, such that the angle 230 is θ=0°. More generally, the angle 230 can be designed between θ=−80° to 80°, depending the geometry and/or characteristics of the vagina 100 and positioning of the uterus.

The gynecological prosthetic 200 has a design that is based on and/or fitted to the anatomical shape of the upper vagina. The gynecological prosthetic 200 can have a better fit to the anatomical shape of the vagina 100 compared to current gynecological prosthetics, in part because its design and flexibility enable it to expand in certain directions and rest on the anchor points in the vaginal canal, for example anterior, posterior and side directions. It is possible to balance pressure applied to the anterior, posterior and side walls. This is an improvement over conventional gynecological prosthetics that may focus pressure on only anterior and posterior walls and are not expandable.

In particular, the gynecological prosthetic 200 implements an expansion mechanism toward specific resting/anchor points to resist displacement, a pressure redistribution mechanism to redirect the pressure from the anterior and posterior walls of the vaginal canal to the lateral walls of the vaginal canal, and a certain angle 230 in the design adapted to the cervical angle formed by the cervical axis and the anterior wall of the vagina. This is an improvement over some conventional gynecological prosthetics that lack such features.

In some implementations, the anchor members 201-204 differ from the connecting links 211 in terms of thickness and/or material. For example, in some implementations, the anchor members 201-204 are formed of a first material and the connecting links 211 are formed of a second material, such that the first material is more rigid than the second material. That being said, it is noted that the second material can be the same as the first material in nature but with different flexibility. The first material is selected such that the anchor members 201-204 are rigid enough to engage with the inside surfaces of the vagina 100. The second material is selected such that the connecting links 211 are compressible to enable the compact state when the gynecological prosthetic 200 is being installed in the vagina 100, and expandable from the compact state after the gynecological prosthetic 200 is installed in the vagina 100. More generally, the gynecological prosthetic 200 can be formed of one or more materials, including but not limited to various types of elastomers such as medical-grade silicone, flexible plastics such as Agilus 30, hard plastic, steel, metal alloys and biocompatible materials such as silicone, etc. In some implementations, the anchor members 201-204 are thicker than the connecting links 211. Other implementations are possible.

In some implementations, the gynecological prosthetic 200 has a silicon layer coating. The silicon layer coating can cover all surfaces of the gynecological prosthetic 200. In other implementations, there is no silicon layer coating. In some implementations, there is no silicone layer coating when the gynecological prosthetic 200 is directly 3D printed using silicone. In some implementations, the gynecological prosthetic 200 has a biocompatible coating other than silicon. In some implementations, the gynecological prosthetic 200 is coated with one or multiple coatings including but not limited to anti-fungus/anti-infection drugs, hormones and ph-balancers, to prevent balance the vaginal acidity and prevent odor-causing bacteria, infections and/or extreme discharge, and/or pigments for aesthetic purposes because silicone is normally clear. In some implementations, the gynecological prosthetic 200 is coated with hormonal drugs to help decrease failure rate of the gynecological prosthetic 200 or can be used as a contraceptive method, letting patients have simultaneous therapies (i.e. hormonal therapy along with prosthetic therapy). Other coatings are possible. In other implementations, there is no such coating.

In some implementations, the connecting links 211 and the anchor members 201-204 are connected in series around a periphery of the gynecological prosthetic 200, and the gynecological prosthetic 200 has a structural mesh 270 connecting the anchor members 201-204 to mitigate deformation of the gynecological prosthetic 200 when installed in the vagina. In other implementations, there is no such structural mesh 270.

In some implementations, the base plate 250 and the upper plate 240 enable a vaginal discharge to pass through the gynecological prosthetic 200. For example, in some implementations, the upper plate 240 is a torus shape through which a vaginal discharge can pass through. Also, in some implementations, the structural mesh 270 of the base plate 250 has holes to enable a vaginal discharge to pass through. Other implementations are possible for allowing a vaginal discharge to pass through the gynecological prosthetic 200. By enabling a vaginal discharge to pass through the gynecological prosthetic 200, the gynecological prosthetic 200 can be used during menstruation. However, alternative implementations are possible in which the gynecological prosthetic 200 does not enable a vaginal discharge to pass through, such that the gynecological prosthetic 200 might be removed for any menstruation.

In some implementations, the gynecological prosthetic 200 has a removing feature (not shown), for example a knob or a string attached to the gynecological prosthetic 200 to collapse and withdraw the gynecological prosthetic 200 compactly at some later time. Other removing features are possible and are within the scope of the disclosure. In some implementations, the gynecological prosthetic 200 is disposable. In some implementations, the gynecological prosthetic 200 is biodegradable when using a material as a wrap for the gynecological prosthetic 200 after its usage that corrupts plastic/silicone and turns the gynecological prosthetic 200 into compost. In other implementations, the gynecological prosthetic 200 is not disposable and can be re-used, for example up to two years or some other suitable time-frame. In some implementations, the gynecological prosthetic 200 is multi-use and can be repeatedly removed and reinserted for up to 29 days or more for example.

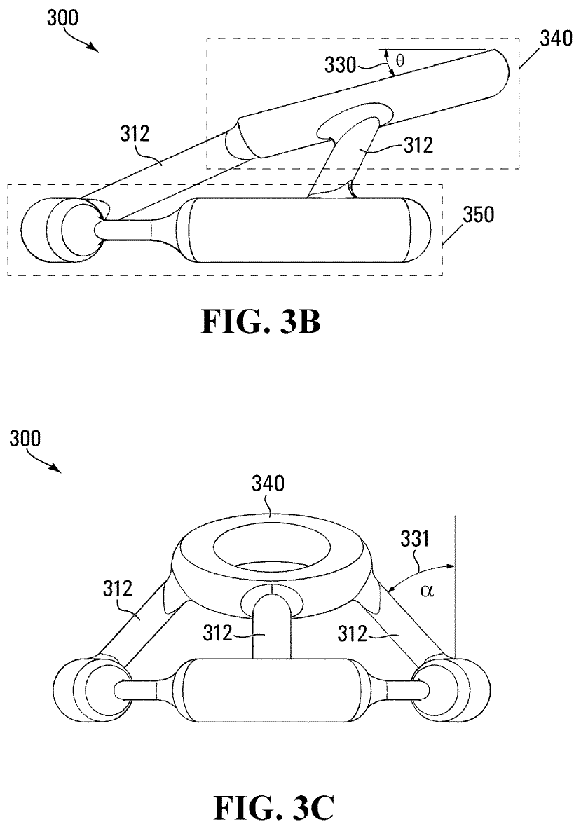

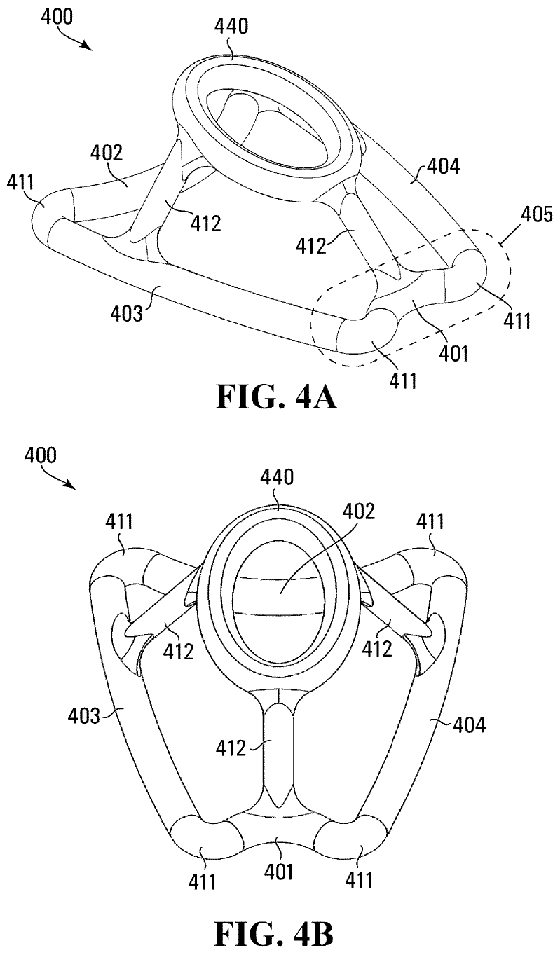

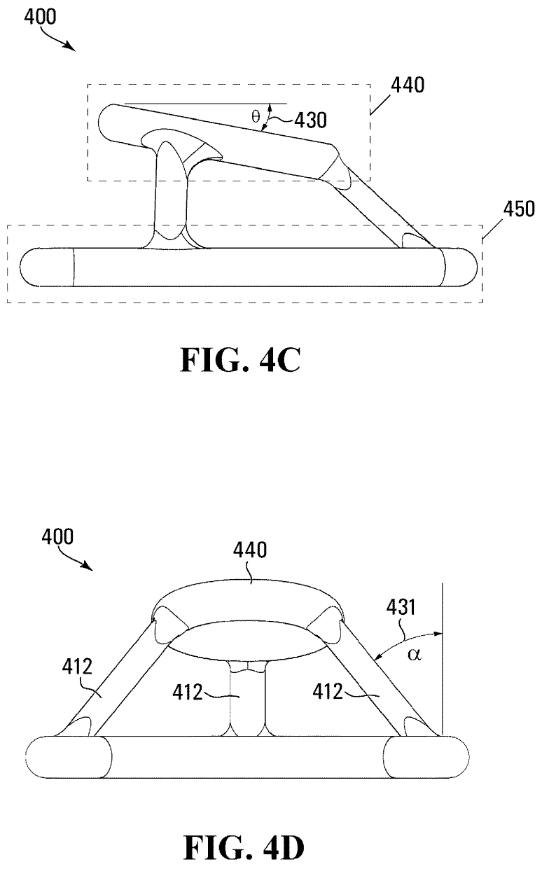

It is to be understood that the gynecological prosthetic 200 of FIGS. 2A to 2E is shown with very specific features for exemplary purposes only. Other gynecological prosthetics are possible and are within the scope of the disclosure. With reference to FIGS. 3A to 3C and FIGS. 4A to 4D, other gynecological prosthetics 300 and 400 are described below which are similar but different from the gynecological prosthetic 200 of FIGS. 2A to 2E. These gynecological prosthetics 300 and 400 are also shown with very specific features for exemplary purposes only. The gynecological prosthetics 200, 300 and 400 are designed to manage uterine prolapse and/or urinary incontinence symptoms. However other gynecological prosthetic designs can be made to focus on rectocele (displacement of rectum), and cystocele (displacement of bladder) where knobs can be added to the side-walls on the base plate to push the rectum or bladder respectively. These gynecological prosthetics 200, 300 and 400 have a dynamic structure where they expand differently and in different directions based on each patient's prolapse and the direction and the place where the pressure is coming from the body to the gynecological prosthetic 200, 300 and 400.

Referring now to FIGS. 3A to 3C, shown are schematics of another gynecological prosthetic 300, in accordance with an embodiment of the disclosure. Similar to the gynecological prosthetic 200 of FIGS. 2A to 2E, the gynecological prosthetic 300 of FIGS. 3A to 3C has a plurality of anchor members 301-303 configured to rest on anchor points within a vagina, and a plurality of connecting links 311 that couple the anchor members 301-303 together. Also, the gynecological prosthetic 300 has an upper plate 340 coupled to a base plate 350 via upper links 312, such that angles 330 and 331 are formed. The gynecological prosthetic 300 is similar to what has already been described above for the gynecological prosthetic 200 of FIGS. 2A to 2E and thus much of the description is not repeated here.

However, there are notable differences between the gynecological prosthetic 300 of FIGS. 3A to 3C and the gynecological prosthetic 200 of FIGS. 2A to 2E. For example, the gynecological prosthetic 300 of FIGS. 3A to 3C has only three anchor members 301-303, and the anchor members 301-303 and the connecting links 311 do not span an entire periphery of the gynecological prosthetic 300. This is because the anchor members 301-303 of the gynecological prosthetic 300 do not include a posterior member. As such, the gynecological prosthetic 300 is designed to avoid or mitigate force applied to the posterior wall of the vagina 100, and to instead apply force to the anterior and side walls of the vagina 100. In another embodiment, there is no anterior member, such that forces are not distributed to the anterior wall of the vagina 100. The gynecological prosthetic 300 of FIGS. 3A to 3C also does not have a structural mesh, although in other implementations it can be equipped with one.

Referring now to FIGS. 4A to 4D, shown are schematics of another gynecological prosthetic 400, in accordance with an embodiment of the disclosure. Similar to the gynecological prosthetic 200 of FIGS. 2A to 2E, the gynecological prosthetic 400 of FIGS. 4A to 4D has a plurality of anchor members 401-404 configured to rest on anchor points within a vagina, and a plurality of connecting links 411 that couple the anchor members 401-404 together. Also, the gynecological prosthetic 400 has an upper plate 440 coupled to a base plate 450 via upper links 412, such that angles 430 and 431 are formed. The gynecological prosthetic 400 is similar to what has already been described above for the gynecological prosthetic 200 of FIGS. 2A to 2E and thus much of the description is not repeated here.

However, there are notable differences between the gynecological prosthetic 400 of FIGS. 4A to 4D and the gynecological prosthetic 200 of FIGS. 2A to 2E. For example, some of the anchor members 401-404 have a concave shape. In particular, the anchor members 401-404 include anterior and posterior members 401 and 402 which are concaved, such that the connecting links 411 may protrude outward to some extent. These connecting links 411 nonetheless operate in a similar manner as the connecting links 211 of the gynecological prosthetic 200 of FIGS. 2A to 2E. The anterior member 401 along with adjacent links 411 create a contour 405 which may provide a better fit against the anterior wall of the vagina 100. Similarly, the posterior member 402 along with adjacent links 411 may provide a better fit against the posterior wall of the vagina 100. The concave shape can mimic the vaginal canal's shape as much as possible and prevent the gynecological prosthetic 400 from blocking the urethra in the anterior part and the rectum in the posterior part. The gynecological prosthetic 400 of FIGS. 4A to 4D also does not have a structural mesh, although in other implementations it can be equipped with one.

After conducting research and development on pelvic floor characteristics, the gynecological prosthetics 200, 300 and 400 disclosed herein have been designed with all relevant information of the pelvic floor and muscles in mind.

In another embodiment, there is provided a gynecological prosthetic configured to rest on anchor points within a vaginal canal. The gynecological prosthetic need not resemble the gynecological prosthetics 200, 300 and 400 depicted and described above. In some implementations, the gynecological prosthetic has geometry and/or materials designed based on a cervical angle.

In some implementations, the geometry and/or materials of the gynecological prosthetic are designed based on at least some of:
abdominal pressure;
intra-vaginal pressure;
pressure from pelvic floor muscles (PFM);
atmospheric pressure (AP);
pressure from bladder towards vaginal canal;
pressure from rectum towards vaginal canal;
intra-abdominal pressure including both transvaginal and transrectal pressure;
intravesical pressure;
intra-abdominal pressure including transvaginal pressure;
distance between ischial spines;
distance between ischial spines and coccyx;
distance between ischial spines and pubic symphysis;
anterior fornix distance;
posterior fornix distance;
cervical size; and
cervical angle.

### Method for Custom-Designing a Gynecological Prosthetic

It is to be understood that the vagina 100 as depicted in FIGS. 1A and 1B is merely exemplary and that varying shapes and sizes are not only possible but are expected among a population of women based on genetics, ethnicity, parity, age, Body Mass Index (BMI), and other factors. A woman's vagina is generally unique in terms of exact shape and size. As such, it is generally advantageous to custom-design a gynecological prosthetic for a woman's vagina so that the gynecological prosthetic may suitably fit. A gynecological prosthetic can be customized in terms of shape and/or size according to a specific patient.

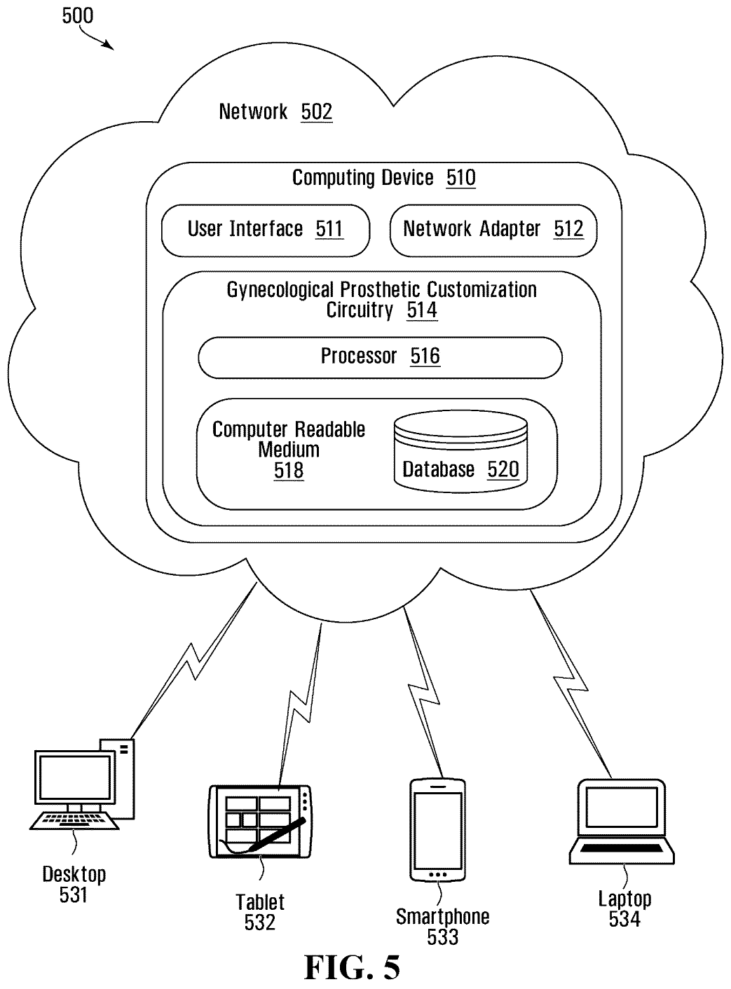

Referring now to FIG. 5, shown is a block diagram of a gynecological prosthetic customization system 500 having a computing device 510. The computing device 510 is cloud-based and has gynecological prosthetic customization circuitry 514. In some implementations, the computing device 510 also has a user interface 511 for interacting with a user, and/or a network adapter 512 for communicating with client computing devices 531-534 over a network 502. The computing device 510 can have additional components, but these are not shown for simplicity. The client computing devices 531-534 can for example include a desktop computer 531, a tablet computer 532, a smartphone 533, a laptop 534, and/or any other appropriate client computing devices.

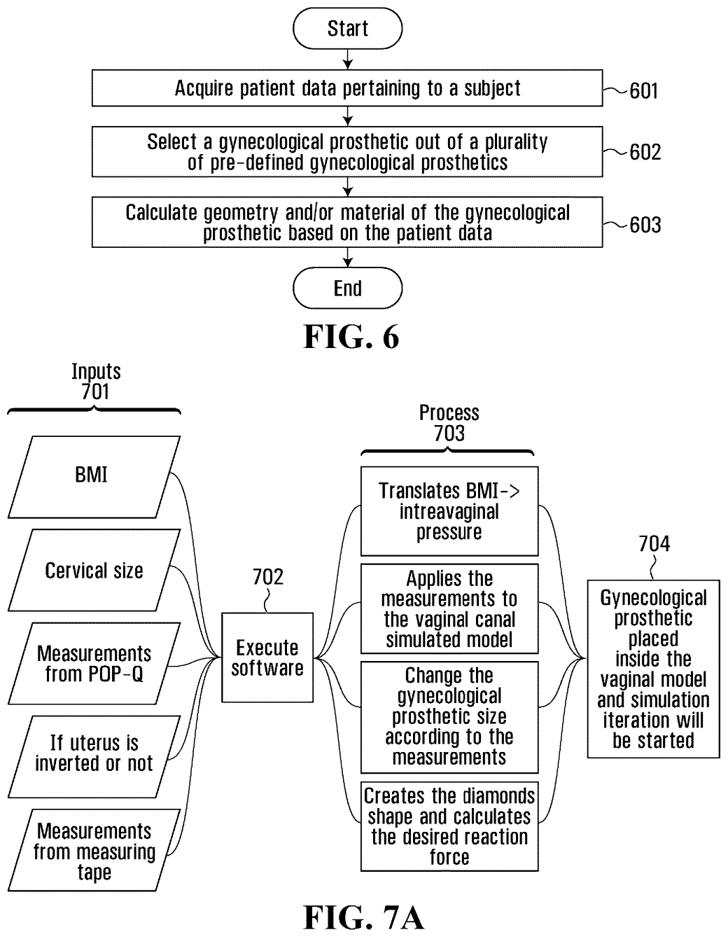

The gynecological prosthetic customization circuitry 514 of the computing device 510 operates to customize a gynecological prosthetic. Such operation will be described below with reference to FIG. 6, which is a flowchart of a method of custom-designing a gynecological prosthetic. Although the method of FIG. 6 is described below with reference to the computing device 510 in the gynecological prosthetic customization system 500 shown in FIG. 5, it is to be understood that the method of FIG. 6 is applicable to other systems. In general, the method of FIG. 6 is applicable to the computing device 510 in any appropriately configured system.

At step 601, the computing device 510 acquires patient data pertaining to a subject, for example from the user interface 511 and/or one or more of the client computing devices 531-534. A patient's vaginal canal can be measured by a physician using manual measurements, for example by using a POP-Q interactive assessment tool. The computing device 510 can receive these measurements, BMI values, and additional patient-specific metrics.

At step 602, the computing device 510 selects a gynecological prosthetic out of a plurality of pre-defined gynecological prosthetics. Each pre-defined gynecological prosthetic has been designed using pre-clinical data and is configured to expand differently and in different directions based on received pressure from surrounding tissues and organs. The pre-defined gynecological prosthetics can for example include the gynecological prosthetics 200, 300 and 400 that have been depicted and described herein, and/or other gynecological prosthetics. The computing device 510 can select which one of these predefined gynecological prosthetics would be most suitable, based on the patient data.

At step 603, the computing device 510 calculates geometry and/or material of the gynecological prosthetic based on the patient data that has been acquired. In this way, the gynecological prosthetic can be customized based on characteristics of the subject, such as a size of the upper vagina for example, and the gynecological prosthetic can be tailored specifically to the subject's anatomy and body habitus. In doing so, the gynecological prosthetic isn't necessarily designed from scratch, because it is based on the pre-defined gynecological prosthetic that has been selected. The patient data can be fitted onto findings and predetermined gynecological prosthetic geometry and materials.

In some implementations, the computing device 510 maintains pre-clinical data, such that the selecting and the calculating steps are based on both the pre-clinical data and the patient data. The pre-clinical data can for example include existing CT scans, MRI images, ultrasound scans, and other existing patient data/information that could influence the geometry of the design.

In some implementations, the computing device 510 generates a vaginal canal model based on the patient data, and simulates the gynecological prosthetic in the vaginal canal model to evaluate suitability of the gynecological prosthetic in accordance with a target function. The target function is a mathematical function that is used to calculate suitability based on a defined criteria. The defined criteria can for example include reducing size of the gynecological prosthetic while simultaneously enabling enough outward forces by the anchor members to avoid the gynecological prosthetic from being dislodged and while reducing a maximum force on any one surface. Other defined criteria are possible. The computing device 510 can calculate the geometry and/or material of the gynecological prosthetic in a manner that adjusts the geometry and/or material of the gynecological prosthetic to enhance the suitability of the gynecological prosthetic based on the target function. An Artificial Neural Network (ANN) or other Machine Learning (ML) method can be employed in this regard. ANN and ML methods can be used to find solutions by tweaking variables in such a way that reduces or increases a target function until convergence at a solution.

An automated finite element analysis (FEA) study has been developed. During an automated FEA study, called parametric study, simulation software (Finite Element Analysis Software and topology optimization) can be coupled with ML algorithms such as ANN algorithms to find a suitable performing and topologically suitable design. The customized design is based on the patient's anatomy and is capable of distributing the forces and stresses in the direction of supporting vaginal tissues. Topology optimization can be performed to reduce weight of the gynecological prosthetic without compromising functionality. In some implementations, calculation of material is part of the topology optimization in which how much material is used can be designed, but the material itself may be the same for everyone. However, the material can be designed in other implementations depending on ability to directly 3d print biocompatible silicone.

There are many possibilities for the gynecological prosthetic customisation circuitry 514 of the computing device 510. In some implementations, the gynecological prosthetic customisation circuitry 514 includes a processor 516 that executes software, which can stem from a computer readable medium 518. In some implementations, the computer readable medium 518 also has a database 520 for storing data as described herein. However, other implementations, besides software implementations, are possible and are within the scope of this disclosure. It is noted that other implementations can include additional or alternative hardware components, such as any appropriately configured FPGA (Field-Programmable Gate Array), ASIC (Application-Specific Integrated Circuit), and/or microcontroller, for example. More generally, the gynecological prosthetic customisation circuitry 514 of the computing device 510 can be implemented with any suitable combination of hardware, software and/or firmware.

According to another embodiment of the disclosure, there is provided a non-transitory computer readable medium having recorded thereon statements and instructions that, when executed by the processor 516 of the computing device 510, implement a method as described herein. The non-transitory computer readable medium can be the computer readable medium 518 of the computing device 510 shown in FIG. 5, or some other non-transitory computer readable medium. The non-transitory computer readable medium can for example include an SSD (Solid State Drive), a hard disk drive, a CD (Compact Disc), a DVD (Digital Video Disc), a BD (Blu-ray Disc), a memory stick, or any appropriate combination thereof.

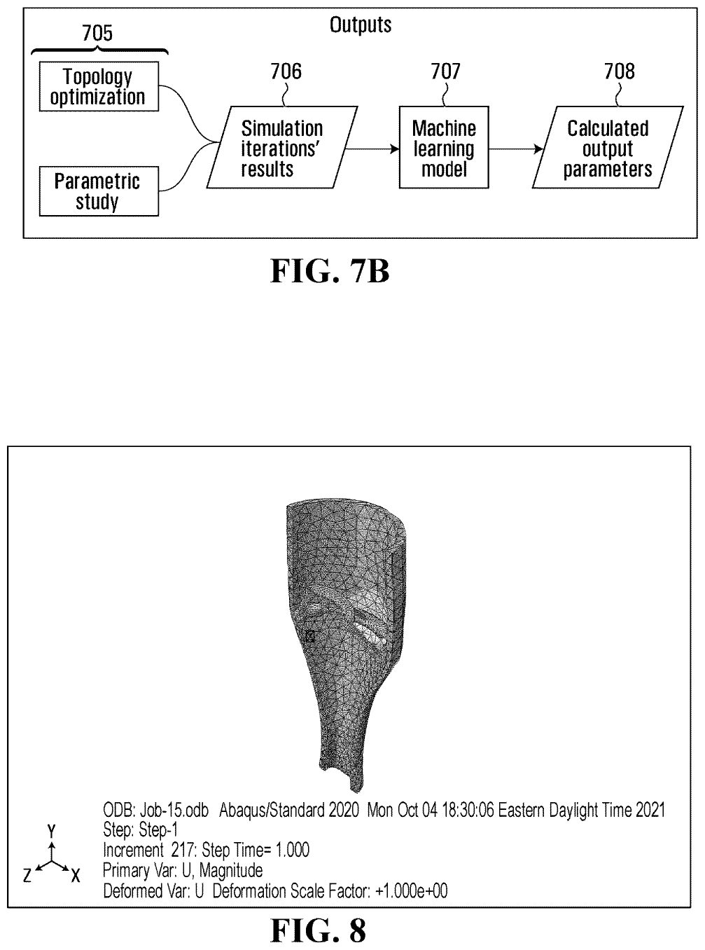

Referring now to FIGS. 7A and 7B, shown is a flowchart of another method of custom-designing a gynecological prosthetic. Although the method of FIGS. 7A and 7B is described below with reference to the computing device 510 in the gynecological prosthetic customization system 500 shown in FIG. 5, it is to be understood that the method of FIGS. 7A and 7B is applicable to other systems. In general, the method of FIGS. 7A and 7B is applicable to the computing device 510 in any appropriately configured system.

At step 701, the computing device 510 acquires patient data pertaining to a subject, for example from the user interface 511 and/or one or more of the client computing devices 531-534. The patient data can include BMI, cervical size, measurements from POP-Q, whether uterus is inverted, and measurements from a measuring tape or a Caliper. However, additional and/or alternative patient data is possible.

At step 702, the computing device 510 executes software to process the patient data. The software can perform various processes at step 703, such as translating BMI to intravaginal pressure, applying measurements to a vaginal canal simulated model, changing a gynecological prosthetic size according to the measurements and the distance of the ischial spines to each other and to the pubis and coccyx, and creating a diamond shape and calculating a desired reaction force and its direction. However, additional and/or alternative processes are possible.

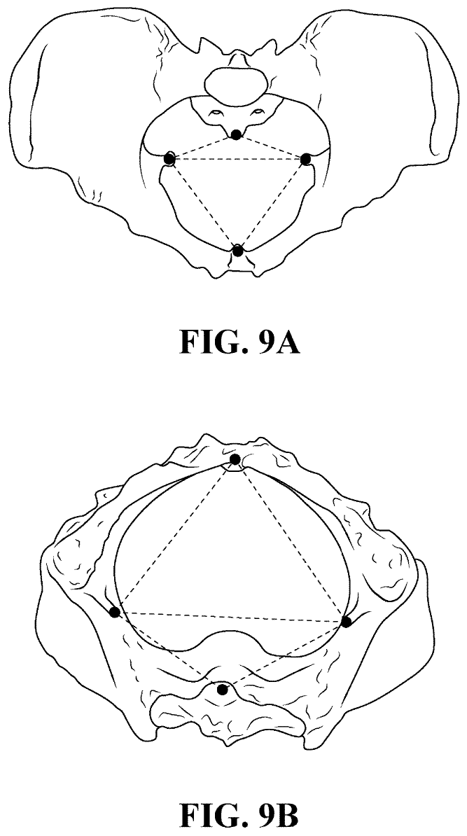

Regarding steps 702 and 703, FIG. 8 is a schematic of the vaginal canal simulated model and show where a gynecological prosthetic may rest. Also, FIGS. 9A and 9B are schematics of a pelvic bony structure including a top view (FIG. 9A) and a bottom view (FIG. 9B). Distances between ischial spines, coccyx and pubic symphysis are shown. These distances can be determined and used in calculating the diamond shape (or two triangles) and the desired reaction force and its direction. The vertices of the diamond shape include the ischial spines, pubic and coccyx. This creates a boundary of a 3D modeling space. The diamonds' sides (including but not limited to one or a combination of multiple sides) can be where the gynecological prosthetics side sections can rest on. In some implementations, there is provided image recognition to recognize the ischial spines.

Referring back to FIG. 7, at step 704, the computing device 510 places a gynecological prosthetic inside the vaginal canal simulated model, and simulation iterations are started. The outputs of the model can include but are not limited to: displacements, reaction forces, total deformations, stress distribution, etc. The iterations can include steps 705 to 708, which can involve topology optimization and parametric study (step 705) as described above, simulation iteration results (step 706), machine learning to evaluate and modify the gynecological prosthetic based on the simulation iteration results (step 707), and calculating output parameters based on the machine learning (step 708).

After completion of the iterations, for example upon steady-state convergence of the output parameters, the output parameters for the gynecological prosthetic should result in a gynecological prosthetic that should suitably fit the subject. The gynecological prosthetic can then be manufactured, for example using 3D printing or by other manufacturing means. The gynecological prosthetic can be directly 3D printed, or a mold of its shape can be 3D printed and then coated. In some implementations, the gynecological prosthetic is manufactured using a medical-grade silicone and/or a biocompatible one or combination of multiple materials or to be molded and/or cast with the mentioned materials.

In some implementations, the gynecological prosthetic is 3D printed using PolyJet technology to combine at least two polymers in the gynecological prosthetic, and then the gynecological prosthetic is coated with a medical grade silicon. PolyJet technology is a 3D printing technology that can produce smooth, accurate parts, prototypes and tooling. With microscopic layer resolution and high accuracy, it can produce thin walls and complex geometries using a wide range of materials. In some implementations, the at least two polymers includes Agilus30 and Vero, which can be used to create flexible rubbers with varying and designed flexibility. Other polymers are possible. Note that this manufacturing method can be used to manufacture other gynecological prosthetics besides the gynecological prosthetics 200, 300 and 400 that have been depicted and described herein. More generally, the manufacturing method can be used to manufacture any suitable gynecological prosthetic.

After the gynecological prosthetic is manufactured, it can be sent to a clinician for validation. In some implementations, for ongoing management, a digital health platform can be provided by the computing device 510 so that clinicians can manage the patients' treatment plans etc. The clinicians can access the digital health platform through the client computing devices 531-534. The digital health platform can be implemented using cloud software on the computing device 510 to show treatment plans online and support digital health management.

This method can enable an intravaginal customized gynecological prosthetic to treat feminine pelvic organ prolapse and urinary/fecal incontinence. Clinicians can obtain patient-specific measurements of the vaginal canal (through any available method they use). They can input the measurements into the computing device 510. These measurements can be fit to the vaginal canal model and the design of the gynecological prosthetic will adapt to this vaginal canal's anatomical characteristics as described above.

There are many possibilities for the patient data. Specific example details for the patient data, including measuring tools for measuring the patient data, are provided in Table 1 below. It is to be understood that Table 1 is very specific and is provided merely for exemplary purposes.

TABLE 1
Patient Data and Measuring Tools for the Same

| Measurement | Measurement | | | |
|---|---|---|---|---|
| Type | Name | Definition | Example Usage | Measurement Tools |
| POP-Q | gh | Genital Hiatus | The probably of the | POP-Q Interactive |
| | | | displacement of the | Assessment Tool |
| | | | gynecological | |
| | | | prosthetic through the | |
| | | | vaginal entrance | |
| | TvL | Total Vaginal | Shows the probability | POP-Q Interactive |
| | | Length | of the displacement of | Assessment Tool |
| | | | the gynecological | |
| | | | prosthetic from the | |
| | | | vaginal Canal as well | |
| | | | as the space of the | |
| | | | vaginal canal that can | |
| | | | be used by the | |
| | | | gynecological | |
| | | | prosthetic | |
| | Aa | Anterior | Coupled with the | POP-Q Interactive |
| | | Wall | length of the posterior | Assessment Tool |
| | | | wall shows the | |
| | | | approximate cervical | |
| | | | angle | |
| | Ba | Anterior | Anterior Prolapse | POP-Q Interactive |
| | | Prolapse | degree | Assessment Tool |
| | | Degree | | |
| | Ap | Posterior | Coupled with the | POP-Q Interactive |
| | | Wall | length of the anterior | Assessment Tool |
| | | | wall shows the | |
| | | | approximate cervical | |
| | | | angle | |
| | Bp | Posterior | Posterior prolapse | POP-Q Interactive |
| | | Prolapse | degree | Assessment Tool |
| | | Degree | | |
| | Pb | Perineal | How strong is the | POP-Q Interactive |
| | | Body | perineal body and | Assessment Tool |
| | | | pelvic floor muscles | |
| | | | and the probability of | |
| | | | the displacement of the | |
| | | | gynecological | |
| | | | prosthetic out of the | |
| | | | vaginal canal | |
| | D | Posterior | Apical Prolapse Degree | POP-Q Interactive |
| | | Fornix | | Assessment Tool |
| | C | Cervix/Cuff | Apical Prolapse Degree | POP-Q Interactive |
| | | | | Assessment Tool |
| BMI | BMI | Body Mass | | Weighing Scale, Tape |
| | | Index | | Measure, Calculator, |
| | | | | BMI Table |
| | IAP/BMI | Intra- | Translate it with the | Software |
| | | Abdominal | formula: IAP/BMI = | |
| | | Pressure (both | 0.76(P *< 0.0001) | |
| | | transvaginal | CmH2O to obtain the | |
| | | and transrectal) | intra abdominal | |
| | | | pressure(both | |
| | | | transvaginal and | |
| | | | transrectal) | |
| | IVP/BMI | Intravesical | Translate it with the | Software |
| | | Pressure | formula: IVP/BMI = | |
| | | | 0.71(P < 0.0001) | |
| | | | CmH2O to obtain the | |
| | | | intervesical pressure | |
| | IAP/BMI | Intra- | Translate it with the | Software |
| | | Abdominal | formula: IAP/BMI = | |
| | | Pressure | 0.73(P < 0.0001) | |
| | | (Transvaginal) | CmH2O to obtain the | |
| | | | transvaginal pressure | |
| Manual or | N/A | Distance | Defining the triangle | Measuring Tape or |
| Digital Exam | | Between | and diamond shape | Caliper Tool |
| Measurements | | Ischial Spines | references (FIGS. 9A | |
| | | | and 9B) | |
| | N/A | Distance | Defining the triangle | Measuring Tape or |
| | | Between | and diamond shape | Caliper Tool |
| | | Ischial Spines | references (FIGS. 9A | |
| | | and Cocyx | and 9B) | |
| | N/A | Distance | Defining the triangle | Measuring Tape or |
| | | Between | and diamond shape | Caliper Tool |
| | | Ischial Spines | references (FIGS. 9A | |
| | | and Pubic | and 9B) | |
| | | Symphysis | | |
| | N/A | Anterior | Determines the size of | Measuring Tape or |
| | | Fornix | the anterior part of the | Caliper Tool |
| | | Distance | gynecological | |
| | | | prosthetic | |
| | N/A | Posterior | Determines the size of | Measuring Tape or |
| | | Fornix | the posterior part of the | Caliper Tool |
| | | Distance | gynecological | |
| | | | prosthetic | |
| | N/A | Cervical Size | Diameter of the cervix | Cervical Sizer Tool |
| | | | that goes to the hole of | |
| | | | the gynecological | |
| | | | prosthetic | |

*P: Pearson correlation coefficient.

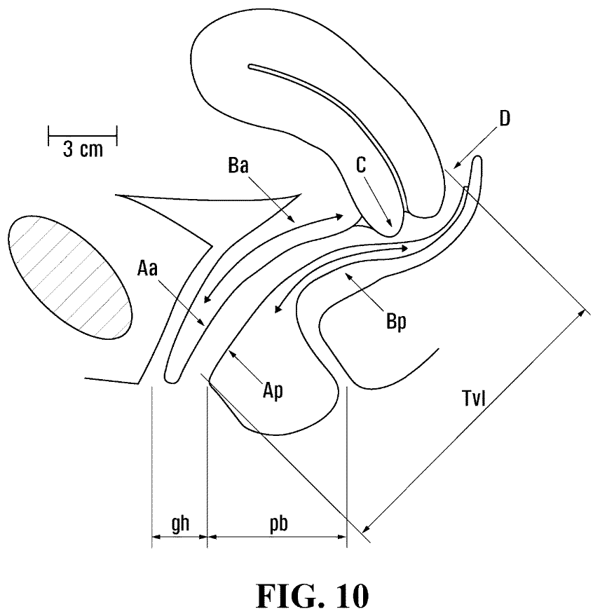

The “POGP-Q Interactive Assessment Tool” referenced in Table 1 refers to an objective, site-specific system for describing, quantifying, and staging pelvic support in women as is known to those skilled in the art. FIG. 10 is a schematic with variables measured using the POP-Q measurement system. The variables are listed below.

TABLE 2
Variables Measured using POP-Q

| Point | Description | Range of Values |
|---|---|---|
| Aa | Anterior vaginal wall 3 cm proximal | −3 cm to +3 cm |
| | to the hymen | |
| Ba | Most distal position of the remaining | −3 cm to +tvl |
| | upper anterior vaginal wall | |
| C | Most distal edge of cervix or vaginal | |
| | cuff scar | |
| D | Posterior fornix (N/A if post-hysterectomy) | |
| Ap | Posterior vaginal wall 3 cm proximal | −3 cm to +3 cm |
| | to the hymen | |
| Bp | Most distal position of the remaining | −3 cm to +tvl |
| | upper posterior vaginal wall | |

Genital hiatus (gh) - Measured from middle of external urethral meatus to posterior midline hymen
Perineal body (pb) - Measured from posterior margin of gh to middle of anal opening
Total vaginal length (tvl) - Depth of vagina when point D or C is reduced to normal position

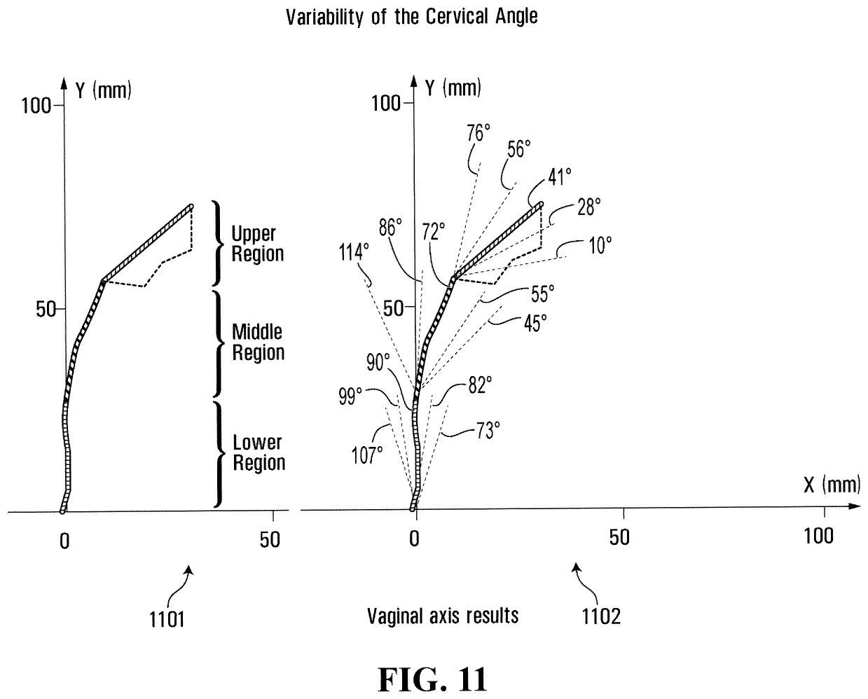

The POP-Q measurement system can be used to approximate the cervical angle 121. FIG. 11 is a graph of the cervical angle 121 vs. position within the vagina 100. On the left side 1101, the cervical angle 121 is indicated for various positions (i.e. lower region, middle region, and upper region) within the vagina 100. On the right side 1102, for each position, a range of values is provided for the cervical angle 121 in percentile form based on a sampling of many vaginas, because vaginas can vary in terms of size and shape. In particular, for each position within the vagina 100, five cervical angles 121 are provided for 5^{th} percentile, 25^{th} percentile, 50^{th} percentile, 75^{th} percentile, 95^{th} percentile. For example, for the upper region, the cervical angles 121 include 10° for 5^{th} percentile, 28° for 25^{th} percentile, 41° for 50^{th} percentile, 56° for 75^{th} percentile, and 76° for 95^{th} percentile. Such variability in the cervical angle 121 bolsters the usefulness of obtaining patient-specific measurements with a view of customizing gynecological prosthetics. As shown by FIG. 11, the cervical angle 121 in the upper region varies between θ=10° to θ=76° for most vaginas (i.e. excluding bottom 5% and top 5%), but as noted above a gynecological prosthetic can be designed for any cervical angle between θ=−80° to 80°.

The “Weighing Scale, Tape Measure, Calculator, BMI Table” referenced in Table 1 refers to any suitable means for assessing BMI of the subject as would be known to those skilled in the art.

The “Software” referenced in Table 1 refers to the software that calculates geometry and/or material of the gynecological prosthetic based on patient data. See for example FIGS. 7A and 7B. The software can take inputs from clinicians. Clinicians can fill in the software with their obtained measurements including the digital measurements and POP-Q (shown in Table 1). These measurements may include vaginal hiatus, vaginal canal's length, depth and width, the position of the anterior and the posterior fornix, width of the upper vagina, the width of the anterior and posterior fornix, the distance from posterior fornix to the pubis, distance between the ischial spines, position of the bones and other organs or tissues including but not limited to the sacrum, Pubic, ischial spines and the distance between them.

These measurements along with other inputs including but not limited to BMI of the patient can be inserted into the software and be transferred to a mechanical model of the vagina. The BMI will be translated to the vaginal pressure. This vaginal pressure will be applied to the vaginal canal model in a simulation software/environment. The vaginal pressure is up to 106 kPa and the Valsalva pressure is up to 58.87 kPa. In some implementations, the customized gynecological prosthetic should not exceed these pressures+/−20 kPa. The algorithm then will adapt our predetermined design of the gynecological prosthetic or any other design suitable for the patient and determine the final gynecological prosthetic according to the vaginal model and these input measurements.

The “Measuring Tape or Caliper Tool” referenced in Table 1 can be a measuring tape which is a retractable finger size measuring tape that can be placed on one or more fingers and can be used to measure any distance and dimensions inside the pelvic cavity. One side of this tape can be fixed on a desired point and the other side can be expanded and locked in any desired point. Additionally, or alternatively, the “Measuring Tape or Caliper Tool” referenced in Table 1 can be a caliper that can be inserted and placed on the ischial spines and show the distance between them.

The “Cervical Sizer Tool” referenced in Table 1 refers to a tool for measuring the cervix as would be known to those skilled in the art.

In some implementations, the inputs from POP-Q (e.g. vaginal hiatus, vaginal canal's length and depth, the position of the anterior and the posterior fornix) and digital/manual measurements (e.g. width of the upper vagina, width of the anterior, and posterior fornix, the distance from posterior fornix to the pubis, distance between the ischial spines, distance from symphysis pubis to ischial spines, and distance from coccyx to ischial spines) are transferred to a mechanical model of the vagina.

There are many possibilities for the geometry and/or material of the gynecological prosthetic that can be calculated. Specific example possibilities for the geometry of the gynecological prosthetic are provided in Table 3 below. It is to be understood that Table 3 is very specific and is provided merely for exemplary purposes.

TABLE 3
Gynecological Prosthetic and Vaginal Dimensions

| | |
|---|---|
| Name of organ or gynecological | |
| prosthetic component | Dimension Range |

| | | |
|---|---|---|
| Anterior Fornix Length (e.g. the anterior | 8-35 | mm |
| member 201 of the gynecological prosthetic | | |
| 200 shown in FIGS. 2A to 2E) | | |
| Posterior Fornix Width | 26-82.8 | mm |
| Posterior Fornix Length (OS to posterior | 12.5-48.4 | mm |
| fornix in the same plane) | | |
| Gynecological Prosthetic Length | 40.8-95 | mm |

| | |
|---|---|
| Cervical Angle (tilt of an upper plate | θ = −80° to 80° |
| relative to a base plate) | |

| | | |
|---|---|---|
| Middle Region Vagina Width (3 cm above | 15.8-36.7 | mm |
| the introitus) | | |
| 1 cm upper than the middle region width | 18.7-37 | mm |
| Vaginal Surface | 16-34 | cm² |

After the vaginal canal model is created based on these measurements, dimensions of the gynecological prosthetic can be calculated for both the upper plate and the bottom plate: length, width, height, angles of the upper plate and the upper links, length of the upper links, amount of material needed and direction of reaction force. For example, the cervical angle referenced in Table 3 can be used to design a tilt of an upper plate relative to a base plate as previously described.

In some implementations, the mechanical and structural design is simulated using a numerical method, which is an approach of solving mathematical or physical equations using computers. This can be done by converting differential equations defined in continuous space and time to a large system of equations in discretized domain. See for example Brian H. Hahn, Daniel T. Valentine, Chapter 14—Introduction to Numerical Methods, Editor(s): Brian H. Hahn, Daniel T. See also Valentine, Essential MATLAB for Engineers and Scientists (Sixth Edition), Academic Press, 2017, Pages 295-323, ISBN9780081008775, https://doi.org/10.1016/B978-0-08-100877-5.00016-5.

In some implementations, digital measurements are transformed into data usable by a predetermined shape based on initial design parameters of the patient specific gynecological prosthetic and the predetermined shape will change to the mentioned measurements. In some implementations, the vaginal model is parametric and it will depend and change based on each patient's specific measurements. The parametric design can produce a customized design based on each patient's specific measurements. In some implementations, the parametric design can be imported to finite element software to simulate the deformation of the gynecological prosthetic and the vagina under relevant environment in order to find suitable geometry. In some implementations, the vaginal model employs topology optimization in order to create a lighter gynecological prosthetic with the same functionality (i.e. reduce weight of the gynecological prosthetic without compromising functionality).

In some implementations, the inputs include: BMI, POP-Q measurements, Digital (manual) measurements (as shown in Table 1). In some implementations, the inputs may include any available data inputs obtained from clinicians including but not limited to any kind of medical imaging including but not limited to MRI, CT scans, ultrasound, or any device used for pelvimetry, or obtaining vaginal measurements. In some implementations, the BMI is translated into any form of vaginal pressure according to the correlation between BMI and intravaginal pressure or force.

In some implementations, the mentioned measurements (shown in Table 1) are used in an AI/machine learning model. This model can be trained based on this data. The mechanical parameters of the gynecological prosthetic (either predetermined or not) can be designed by this algorithm resulting in finding a suitable model of the gynecological prosthetic for each particular patient. To find the suitable customized design ML/AI models can be trained on the data from measurements and mathematical models (e.g. Finite element analysis). ML/AI models can predict the suitable input design parameters (including but not limited to shape, dimension, angles, materials) to reach the best performing customized design. Algorithms can be based on Neural Networks and/or other optimization algorithms. In some implementations, parameters and hyperparameters are used in the AI. Hyperparameters of the ML models can be tweaked using different optimization algorithms. For example, in neural networks, these involve a number of layers and number of iterations, etc.

In some implementations, the geometry of the gynecological prosthetic that can be calculated includes at least some of length of anterior anchor member, length of posterior anchor member, gynecological prosthetic length, gynecological prosthetic width, an angle of upper plate relative to base plate, length of connecting links, length of upper links, size of the upper plate, position of the upper plate in relation to the base plate, length of side anchor members, dimensions of the upper plate, thickness of connecting links, and thickness of the upper links. Other implementations are possible.

In some implementations, there is provided an anatomical model of the vaginal canal that includes the position of the ischial spines, cervical angle which is the angle between the uterus and vaginal axis or between the cervical axis and vaginal axis. This cervical angle can vary between 10° and 76° (or θ=−80° to 80° as described earlier). Also, the middle region of the vaginal axis can vary between 45° and 114°. The lower region of the vaginal axis can vary between 73° and 107°.

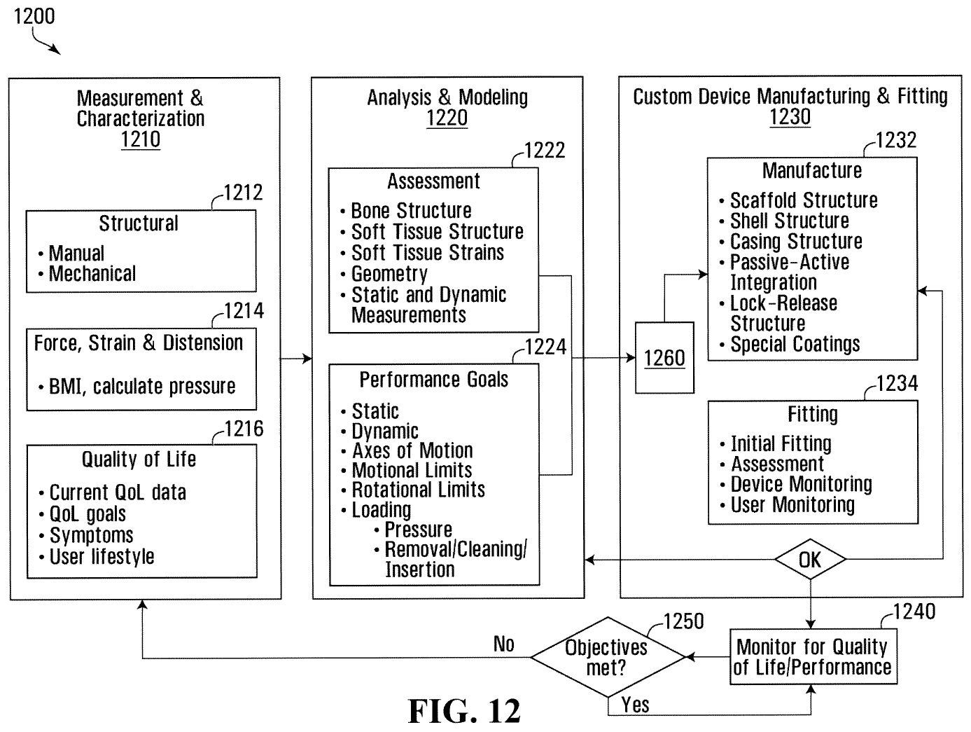

Referring now to FIG. 12, shown is a flowchart of an exemplary process for providing a user with a user specific therapeutic device (USTD), in accordance with an embodiment of the disclosure. The process can reduce a bewildering array of USTD types and dimensions to a single USTD option without significant effort from either the patient or the clinician. Accordingly, at step 1210 the process begins with the step of Measurement and Characterisation (M&C) 1210 before progressing to Analysis and Modelling (A&M) 1220 and Custom Device Manufacturing and Fitting (CUDEMAF) 1230 wherein the patient (user) is now provided and fitted with a custom USTD. Next, the process proceeds to step 1240 wherein ongoing monitoring of quality of life (QoL) and performance of the USTD wherein a decision process 1250 may determine whether the objectives of the USTD are being met or still being met on an ongoing basis and hence determine whether monitoring should continue or whether the process should begin again with step 1210. An ongoing monitoring and cyclic process may be appropriate for a variety of USTD use cases including, but not limited to, changing physical characteristics of the user, changing physiology of the user, and degradation of the USTD. Accordingly, as depicted M&C 1210 comprises three sub-processes, these being, Structural 1212, Force, Strain and Distension 1214, and Quality of Life 1216.

Within embodiments of the disclosure, the custom USTD may be employed in combination with other therapies and/or pharmaceutical coatings etc. in order to combine a custom USTD with regenerative medicine. Accordingly, within other embodiments of the disclosure, a USTD according to an embodiment of the disclosure may exploit an energy delivery system such as infrared irradiation or ultraviolet irradiation for example. A custom USTD may also be employed in conjunction with other medical procedures and/or treatment regimens including, for example, exploitation of stem cells.

Structural 1212 may comprise one or more measurements of the user's anatomy and/or measurements of the user's physical characteristics such that one or more characteristics such as the dimensions of the user's major anatomical structures, anatomical geometry, etc. are defined. Examples of such measurements have been described above and are not repeated here.

Force, Strain and Distension 1214 may comprise one or more measurements of characteristics of the user's anatomy and/or measurements of the user's physical characteristics, for example to ascertain BMI and calculate pressure based on the same, as previously described. The measurements performed within Structural 1212 and Force, Strain and Distension 1214 may be statically acquired, i.e. with the user sitting/laying/standing within a clinic or another environment and/or dynamically acquired with the user performing one or more routine aspects of their life such as walking, exercising, running, lifting, bending, etc. In contrast to the Structural 1212 and Force, Strain and Distension 1214 the Quality of Life 1216 is an assessment.

Quality of Life (QoL) 1216 may include, but not limited to, current QoL data for the user (patient), QoL goals for the user (patient), symptoms experienced by the user, and user lifestyle. Accordingly, QoL 1216 can establish baseline QoL data which may be employed subsequently for the monitoring, QoL and performance of the USTD once manufactured and employed according to embodiments of the disclosure. Accordingly, for one user a QoL goal may be the elimination of a symptom that occurs only during sexual activity whilst for another it may during a specific exercise, sporting activity, etc. or for another over specific periods of time and/or generally monitored etc. Additionally, the USTD in terms of being permanent, semi-permanent, or temporary is established wherein for temporary use at least the installation/removal means and/or mechanisms are established with the user. For permanent and semi-permanent the installation/removal means are geared primarily to the clinician rather than the user.

In establishing the QoL 1216 a user may employ an application upon a PED and/or FED in order to track the user's (patient's) perceived QoL, to monitor and/or log even occurrences such incontinence, pain, prolapse, gynecological prosthetic fall out, etc. From M&C 1210 the process proceeds to A&M 1220 wherein sub-processes of Assessment 1222 and Performance Goals 1224 are undertaken. Within Assessment 1222 the data obtained within the M&C 1210 step are analysed, for example, through their entry into a human body (anatomical) model (HBM) of the appropriate body region or body regions to define a series of two-dimensional (2D) and/or three-dimensional (3D) perspectives of the user's anatomy as well as other parameters.

Within Performance Goals 1224 the QoL 1216 data is established as specific static and dynamic performance goals for the USTD, axes of motion, motional limits, rotational limits, loading, pressure etc. These aspects may include, but are not limited to, whether the USTD is to address long term or short term issues, whether the USTD is to address recurring episodes together with frequency etc., degree of comfort level desired, will or can the user perform self-removal/cleaning/insertion etc., will this involve periodic visits to a physician or clinic, and will any coatings involve the user periodically dispose of the USTD and use a new USTD. Additionally, additional characteristics may be established with respect to providing an antimicrobial coating, providing controlled pharmaceutical product release(s) such as proteins, regenerative medicine(s), pain killers, or other drugs for the user. These together with the data from Assessment 1222 are employed in defining the custom USTD for the user in terms of physical geometry, e.g. dimensions of any ring structure, knob, support etc. Additionally, the mechanical properties of the custom USTD are defined in respect of the flexibility, dimensional stability, installation/removal means, physical characteristics of the USTD such as smooth/contoured surfaces and/or regions, etc. as well as other aspects such as any locking and/or release mechanisms.

Based upon the established mechanical and physical specifications together with appropriate aspect of the QoL specifications the process in CUDEMAF 1230 proceeds with a sequence comprising Manufacture 1232 and Fitting 1234. The accumulated data from the Analysis & Modelling 1220 as defined within Assessment 1222 and Performance Goals 1224 is coupled to an Artificial Intelligence (AI) Engine 1260 which employs a plurality of algorithms which may exploit one or more approaches including, but not limited to, those based on symbol manipulation, cognitive simulation, logic-based programming, anti-logic programming, natural language processing, knowledge based, sub-symbolic, embodied intelligence, computational intelligence and soft computing, and statistical either individually or in combination such as within methodologies such as the intelligent agent, multiple interacting agents in a multi-agent system, and a hybrid intelligent system.

Within Manufacture 1232 the custom USTD is defined in respect of the materials providing its physical geometry with the desired mechanical properties as well as external characteristics. Accordingly, the custom USTD may be defined by one or more aspects including, but not limited to:
Scaffold structure by dimension(s), material(s) etc.
Shell structure by dimension(s), material(s) etc.
Casing structure by dimension(s), property or properties, material(s).
Passive—active integration such as is USTD passive or does it embed sensor(s), control and/or data logging circuitry, wireless interface(s) etc.
Lock-release structure.
Coatings.

Accordingly, a CAD model is established from which the Manufacture 1232 process is undertaken. Within an embodiment of the disclosure, an initial CAD model may be established by combining three-dimensional (3D) modelling with computational fluid dynamics (CFD), finite element analysis (FEA), and/or multi-organ free-body diagram models. The CAD model may be simplified to reduce the computational power and complexity of the processing applied prior to the AI Engine 1260 executes. The AI Engine 1260 may process based upon this initial pre-processing solely or may apply the preprocessing to a more complete human body (anatomical) model and USTD model in order to define the USTD design, CAD, and materials specifications. Optionally, the pre-processing may be bypassed where appropriate levels of computing resources are available. Accordingly, a USTD as designed and manufactured may range from a passive USTD through to an active USTD, with lock-release structure, anti-microbial coating, and wireless interface for transmitting data logging data relating to the user.

Optionally, within embodiments of the disclosure, the USTD may in addition to sensors include actuators that apply pressure to predetermined regions of the user or may support the user's body motion. Optionally, the USTD may provide controlled release of one or more pharmaceutical agents such as by opening a reservoir to expose said one or more pharmaceutical agents, employ microneedles to inject one or more pharmaceutical agents, etc.

Within Fitting 1234 the custom USTD is provided to the user and either fitted by themselves, e.g. for temporary use USTD that the user will insert/remove as desired, or by a clinician, e.g. semi-permanent or permanent use. At this point one or more assessments may be carried out such as outlined previously in respect of Structural 1212 and/or Force, Strain and Distension 1214 whereby mechanical, imaging, static and/or dynamic assessment etc. are performed to assess the USTD fit against the target design/user physiology etc. This stage may also include device monitoring, e.g. via internal sensors to the USTD, as well as user monitoring, e.g. by personally noting performance of the USTD etc. Based upon these results a determination is made as to whether the USTD meets the initial specifications wherein if the determination is positive then the process proceeds to step 1240. If not, then the process proceeds to loop back to either A&M 1220 or CUDEMAF 1230 according to the nature and/or complexity of the modifications/amendments to be made.

In step 1240 the user employs the USTD on an ongoing basis wherein device monitoring, e.g. via internal sensors to the USTD, as well as user monitoring, e.g. by personally noting performance of the USTD etc. are performed wherein periodically this data is employed in determining whether the objectives for the USTD were met in step 1250. If yes, then the process loops back to step 1240 otherwise it proceeds back to step 1210. For example, a young user may have multiple USTDs within the space of a few years/decade during their childhood, adolescence, puberty, etc. with evolving dimensions and considerations whereas an elderly user may get by with a single adjustment or no adjustment according to their circumstances.

### Gynecological Prosthetic Installation

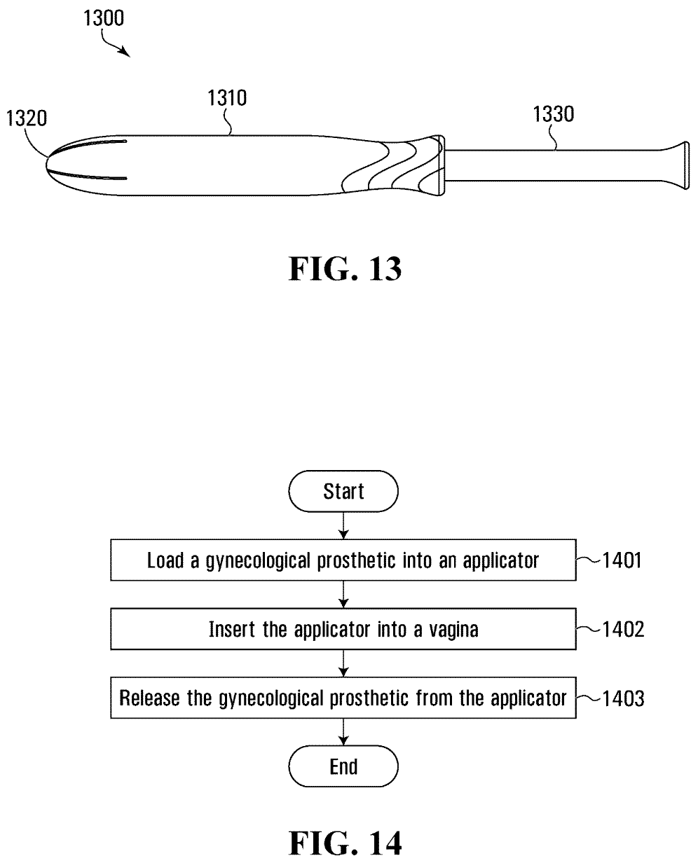

Referring now to FIG. 13, shown is a schematic of an applicator 1300 for installing a gynecological prosthetic as disclosed herein. The applicator 1300 has a tubular body 1310 including a rounded opening 1320 with flaps, and a plunger 1330. Operation of the applicator 1200 will be described below with reference to FIG. 13, which is a flowchart of a method of installing a gynecological prosthetic. The applicator 1300 can be used with a wide variety of gynecological prosthetics, such as any of the gynecological prosthetics 200, 300 and 400 depicted and described herein.

At step 1401, with the plunger 1330 removed from the tubular body 1310, a gynecological prosthetic is loaded into the tubular body 1310. Patients and doctors can fold/collapse the gynecological prosthetic and put it into the applicator 1300 themselves. The gynecological prosthetic is in the compact state while inside the tubular body 1310. The plunger 1330 can then be re-inserted into the tubular body 1310. At step 1402, the applicator 1300 (with the gynecological prosthetic therein) is inserted into a vagina and moved into a suitable position within the vagina. Then, at step 1403, the gynecological prosthetic is released from the applicator 1300 by pushing plunger 1330 further into the tubular body 1310 thereby pushing the gynecological prosthetic out of the rounded opening 1320 and into the vagina. The gynecological prosthetic can be deployed by the applicator 1300 to automatically rest on the anchor points within the vagina based on geometry of the gynecological prosthetic.

Also disclosed is a kit having a gynecological prosthetic as described and/or depicted herein, and an applicator as described and/or depicted herein. Both the applicator and the gynecological prosthetic can be multi-use for 2-years and 29+ days (disposable gynecological prosthetic) respectively. The gynecological prosthetic can also be made to have a shelf life of 2 years if it is not disposable.

Numerous modifications and variations of the present disclosure are possible in light of the above teachings. It is therefore to be understood that within the scope of the appended claims, the disclosure may be practised otherwise than as specifically described herein.

1.-21. (canceled)

22. A method of custom-designing a gynecological prosthetic comprising:
acquiring, by a processor, patient data pertaining to a subject;
selecting the gynecological prosthetic out of a plurality of pre-defined gynecological prosthetics,
wherein each pre-defined gynecological prosthetic is configured to expand differently and in different directions based on received pressure from surrounding tissues and organs; and
calculating, by a processor, geometry of the gynecological prosthetic that has been selected based on the patient data that has been acquired.

23. The method of claim 22, further comprising:
maintaining pre-clinical data, wherein the selecting and the calculating steps are based on both the pre-clinical data and the patient data.

24. The method of claim 22, further comprising:
generating a vaginal canal model based on the patient data; and
simulating the gynecological prosthetic in the vaginal canal model to evaluate suitability of the gynecological prosthetic in accordance with a target function;
wherein calculating the geometry of the gynecological prosthetic comprises adjusting the geometry material of the gynecological prosthetic to enhance the suitability of the gynecological prosthetic based on the target function.

25. The method of claim 24, wherein generating the vaginal canal model comprises:
translating BMI (Body Mass Index) to intravaginal pressure for the vaginal canal model, determining distances of ischial spines to each other and to pubis and coccyx, and
determining a reaction force, and its direction, based on the distances.

26. The method of claim 24, wherein adjusting the geometry of the gynecological prosthetic comprises:
performing a parametric study to determine a topologically suitable design for the gynecological prosthetic; and
performing a topology optimization to reduce weight of the gynecological prosthetic without compromising functionality.

27. The method of claim 22, wherein the patient data comprises a cervical angle and/or data from which the cervical angle can be determined.

28. The method of claim 27, wherein the patient data comprises at least one some of:
abdominal pressure;
intra-vaginal pressure;
pressure from pelvic floor muscles (PFM);
atmospheric pressure (AP);
pressure from bladder towards vaginal canal;
pressure from rectum towards vaginal canal;
intra-abdominal pressure including both transvaginal and transrectal pressure;
intravesical pressure;
intra-abdominal pressure including transvaginal pressure;
distance between ischial spines;
distance between ischial spines and coccyx;
distance between ischial spines and pubic symphysis;
anterior fornix distance;
posterior fornix distance; and
cervical size.

29. The method of claim 22, wherein the patient data comprises at least one of:
genital hiatus, total vaginal length, anterior wall, anterior prolapse degree, posterior wall, posterior prolapse degree, perineal body, posterior fornix, cervix/cuff, body mass index, intra-abdominal pressure for both transvaginal and transrectal, intravesical pressure, intra-abdominal pressure for transvaginal, distance between ischial spines, distance between ischial spines and cocyx, distance between ischial spines and pubic symphysis, anterior fornix distance, posterior fornix distance, cervical size, a size of an upper vagina, and measurements of a vaginal canal;
wherein the at least one of the patient data is acquired by a measuring tape or caliper tool.

30. The method of claim 22, wherein the geometry of the gynecological prosthetic comprises at least one of:
a length of an anterior anchor member of the gynecological prosthetic, a length of a posterior anchor member of the gynecological prosthetic, a gynecological prosthetic length, a gynecological prosthetic width, an angle of an upper plate relative to a base plate of the gynecological prosthetic, a length of a connecting link of the gynecological prosthetic, a length of an upper link of the gynecological prosthetic, a size of the upper plate of the gynecological prosthetic, a position of the upper plate in relation to the base plate of the gynecological prosthetic, a length of a side anchor member of the gynecological prosthetic, dimensions of the upper plate of the gynecological prosthetic, thickness of the connecting link of the gynecological prosthetic, and a thickness of the upper link of the gynecological prosthetic.

31.-36. (canceled)

37. The method of claim 22, further comprising manufacturing the gynecological prosthetic.

38. The method of claim 37, wherein the manufacturing the gynecological prosthetic comprises 3D printing the gynecological prosthetic.

39. The method of claim 37, wherein the manufacturing the gynecological prosthetic comprises 3D printing a mold of the gynecological prosthetic.

40. The method of claim 37, wherein:
the gynecological prosthetic comprises at least two polymers; and
the manufacturing the gynecological prosthetic comprises combining the at least two polymers.

41. The method of claim 37, further comprising coating the gynecological prosthetic.

42. The method of claim 22, wherein the gynecological prosthetic is configured to be expandable from a compact state.

43. The method of claim 42, wherein the gynecological prosthetic is configured to expand differently in different directions when expanding from the compact state.

44. The method of claim 22, wherein the gynecological prosthetic comprises a plurality of anchor members configured to rest on anchor points within a vagina.

45. The method of claim 24, wherein the target function is based on a pre-defined criteria.

46. The method of claim 24, wherein the simulating the gynecological prosthetic in the vaginal canal model to evaluate suitability of the gynecological prosthetic in accordance with the target function comprises iteratively simulating the gynecological prosthetic in the vaginal canal model.

47. The method of claim 26, wherein the performing a parametric study to determine the topologically suitable design for the gynecological prosthetic comprises performing a finite element analysis simulation.
